(19) United States
(12) Patent Application Publication
(10) Pub. No.: US 2006/0223876 A1
(43) Pub. Date: Oct. 5, 2006

(54) Indoloquinone Tumor Radiation Sensitization

(76) Inventors: Randy Burd, Oro Valley, AZ (US); Luigi Lenaz, Newton, PA (US); Shanta Chawla, Irvine, CA (US); Guru Reddy, Irvine, CA (US); Adam P. Dicker, Cherry Hill, NJ (US)

Correspondence Address: PRESTON GATES & ELLIS LLP, 1900 MAIN STREET, SUITE 600, IRVINE, CA, 92614-7319, US

(21) Appl. No.: 11/278,299

(22) Filed: Mar. 31, 2006

Related U.S. Application Data
(60) Provisional application No. 60/667,391, filed on Mar. 31, 2005.

(51) Int. Cl. A61K 31/404

(52) U.S. Cl. 514/418

(57) ABSTRACT

This invention generally relates to Indoloquinone caused tumor radiation therapy sensitization. More specifically, this invention relates to the discovery of indoloquinones as a radiation sensitizer (radiation therapy adjuvant) due to its ability to selectively target hypoxic cells and to damage the DNA of these hypoxic cells. Indoloquinones do so with minimal normal cell and tissue toxicity.

### FIELD OF THE INVENTION

The present invention relates to the sensitization of tumor cells to radiation therapy through the administration of one or more indoloquinones. More specifically, the present invention relates to the sensitization of hypoxic tumor cells to radiation therapy through the administration of one or more indoloquinones.

### BACKGROUND OF THE INVENTION

Radiation therapy (irradiation) is an effective modality for the treatment of a variety of tumor types. Half of all cancer patients will receive radiation therapy during their course of treatment for cancer. While radiation therapy is one of the most widely used treatments for cancer, its effectiveness is reduced when used to treat tumors containing hypoxic cells.

Hypoxic cells are those cells that receive less oxygen than other cells. Typically, because of their low oxygen content, hypoxic cells are more resistant to radiation therapy or chemotherapy. Cells that are more resistant to radiation therapy or chemotherapy can pose a greater danger to cancer patients because of their enhanced ability to survive and spread to other locations in the body.

There are several conventional medicinal agents that are currently used to treat tumors containing hypoxic cells. Included among these agents are Indoloquinones. Apaziquone (EO9), one indoloquinone, is a novel analogue of mitomycin C. Once administered, EO9 is bioreduced by intracellular reductases into active DNA damaging moieties and is believed to target hypoxic cells. Clinical trials have indicated that systemically administered EO9 results in poor drug delivery to tumors, while its local delivery has shown significant anti-tumor activity in various xenograft models. To date, EO9 has only been used as a single treatment agent.

The present invention takes advantage of the discovery that when used in combination with radiation therapy, EO9 and other indoloquinones can sensitize hypoxic cells to the radiation therapy thus contributing to the treatment of a variety of cancers. This administration of indoloquinones in conjunction with radiation therapy offers advantages over the singular administration of either radiation therapy or indoloquinones including EO9.

### BRIEF SUMMARY OF THE INVENTION

The present invention provides for the sensitization of tumor cells to radiation therapy through the administration of indoloquinones. More specifically, the present invention relates to the sensitization of hypoxic tumor cells to radiation therapy through the administration of indoloquinones.

Specifically, one embodiment according to the present invention is a method comprising sensitizing one or more tumors to radiation therapy by administering one or more indoloquinones.

In another embodiment of the methods according to the present invention, the one or more tumors comprise hypoxic cells.

In another embodiment of the methods according to the present invention, the method further comprises administering the one or more indoloquinones to a patient in need thereof wherein the administering comprises systemic and/or local administration and the patient will receive at least two radiation therapies.

In another embodiment of the methods, the administering of the one or more indoloquinones occurs through oral administration. In another embodiment of the methods, the administering of the one or more indoloquinones occurs through intra-tumoral administration. In another embodiment of the methods, the administering of the one or more indoloquinones occurs through intravenous administration. In another embodiment of the methods, the administering of the one or more indoloquinones occurs through intravesical administration. In another embodiment of the methods, the administering of the one or more indoloquinones occurs through intraarterial administration. In another embodiment of the methods, the administering of the one or more indoloquinones occurs through a route selected from one or more of any combination of oral administration, intra-tumoral administration, intravenous administration, intravesical administration and intraarterial administration.

In another embodiment of the methods, the one or more indoloquinones comprise apaziquone (EO9).

In another embodiment of the methods, the administering of the one or more indoloquinones occurs before all radiation therapies of the patient. In another embodiment of the methods, the administering of the one or more indoloquinones occurs before a subset of the radiation therapies of the patient. In another embodiment of the methods, the administering of the one or more indoloquinones occurs after all radiation therapies of the patient. In another embodiment of the methods, the administering of the one or more indoloquinones occurs after a subset of the radiation therapies of the patient. In another embodiment of the methods, the administering of the one or more indoloquinones occurs before and after all the radiation therapies of the patient. In another embodiment of the methods, the administering of the one or more indoloquinones occurs before all radiation therapies of the patient and after a subset of the radiation therapies of the patient. In another embodiment of the methods, the administering of the one or more indoloquinones occurs before a subset of the radiation therapies of the patient and after all radiation therapies of the patient. In another embodiment of the methods, the administering of the one or more indoloquinones occurs before a subset of the radiation therapies of the patient and after a subset of the radiation therapies of the patient.

The present invention also includes compositions. In one composition according the present invention the composition comprises one or more indoloquinones wherein the one or more indoloquinones are directed to be administered in conjunction with a radiation therapy for the treatment of a tumor.

In another embodiment of the compositions, the tumor comprises hypoxic cells.

In another embodiment of the compositions, the one or more indoloquinones are directed to be administered systemically and/or locally. In another embodiment of the compositions, the one or more indoloquinones are directed to be administered through oral administration. In another embodiment of the compositions, the one or more indoloquinones are directed to be administered through intra-tumoral administration. In another embodiment of the compositions, the one or more indoloquinones are directed to be administered through intravenous administration. In another embodiment of the compositions, the one or more indoloquinones are directed to be administered through intravesical administration. In another embodiment of the compositions, the one or more indoloquinones are directed to be administered through intraarterial administration. In another embodiment of the compositions, the one or more indoloquinones are directed to be administered through a route selected from one or more any combination of oral administration, intra-tumoral administration, intravenous administration, intravesical administration and intraarterial administration.

In another embodiment of the compositions, the one or more indoloquinones comprise apaziquone (EO9).

In another embodiment of the compositions, the one or more indoloquinones are directed to be administered to a patient who will receive at least two radiation therapy sessions. In another embodiment of the compositions on, the one or more indoloquinones are directed to be administered before all radiation therapies of the patient. In another embodiment of the compositions, the one or more indoloquinones are directed to be administered before a subset of the radiation therapies of the patient. In another embodiment of the compositions, the one or more indoloquinones are directed to be administered after all radiation therapies of the patient. In another embodiment of the compositions, the one or more indoloquinones are directed to be administered after a subset of the radiation therapies of the patient. In another embodiment of the compositions, the one or more indoloquinones are directed to be administered before and after all the radiation therapies of the patient. In another embodiment of the compositions, the one or more indoloquinones are directed to be administered before all radiation therapies of the patient and after a subset of the radiation therapies of the patient. In another embodiment of the compositions, the one or more indoloquinones are directed to be administered before a subset of the radiation therapies of the patient and after all radiation therapies of the patient. In another embodiment of the compositions, the one or more indoloquinones are directed to be administered before a subset of the radiation therapies of the patient and after a subset of the radiation therapies of the patient.

The present invention also includes dosing regimens. One embodiment of the dosing regimens according to the present invention comprises one or more indoloquinones and instructional information that directs the administration of the one or more indoloquinones in conjunction with a radiation therapy for the treatment of a tumor.

In another embodiment of the dosing regimens, the tumor comprises hypoxic cells.

In another embodiment of the dosing regimens, the instructional information directs the systemic and/or local administration of the one or more indoloquinones. In another embodiment of the dosing regimens, the instructional information directs the administration of the one or more indoloquinones through oral administration. In another embodiment of the dosing regimens, the instructional information directs the administration of the one or more indoloquinones through intra-tumoral administration. In another embodiment of the dosing regimens, the instructional information directs the administration of the one or more indoloquinones through intravenous administration. In another embodiment of the dosing regimens, the instructional information directs the administration of the one or more indoloquinones through intravesical administration. In another embodiment of the dosing regimens, the instructional information directs the administration of the one or more indoloquinones through intraarterial administration. In another embodiment of the dosing regimens, the instructional information directs the administration of the one or more indoloquinones through a route selected from one or more of any combination of oral administration, intra-tumoral administration, intravenous administration, intravesical administration and intraarterial administration.

In another embodiment of the dosing regimens, the one or more indoloquinones comprise apaziquone (EO9).

In another embodiment of the dosing regimens, the instructional information directs the one or more indoloquinones to be administered to a patient who will receive at least two radiation therapy sessions. In another embodiment of the dosing regimens, the instructional information directs the one or more indoloquinones to be administered before all radiation therapies of the patient. In another embodiment of the dosing regimens, the instructional information directs the one or more indoloquinones to be administered before a subset of the radiation therapies of the patient. In another embodiment of the dosing regimens, the instructional information directs the one or more indoloquinones to be administered after all radiation therapies of the patient. In another embodiment of the dosing regimens, the instructional information directs the one or more indoloquinones to be administered after a subset of the radiation therapies of the patient. In another embodiment of the dosing regimens, the instructional information directs the one or more indoloquinones to be administered before and after all the radiation therapies of the patient; before all radiation therapies of the patient and after a subset of the radiation therapies of the patient. In another embodiment of the dosing regimens, the instructional information directs the one or more indoloquinones to be administered before a subset of the radiation therapies of the patient and after all radiation therapies of the patient. In another embodiment of the dosing regimens, the instructional information directs the one or more indoloquinones to be administered before a subset of the radiation therapies of the patient and after a subset of the radiation therapies of the patient.

### BRIEF DESCRIPTION OF THE DRAWINGS

FIG. 1 shows the effect of tumor volume on tumor oxygen tension.

FIG. 2 shows that hypoxia down regulates NQO1 in U87 tumor cells.

FIG. 3 shows that hypoxic tumors have a higher ratio of cytochrome P450 reductase to NQO1.

FIG. 4 shows that fractionated radiation therapy increases the ratio of cytochrome P450 reductase to NQO1 in tumor xenografts.

FIG. 5 shows the average tumor volume after radiation therapy and subsequent EO9 or vehicle administration.

FIG. 6 shows the linear regression and estimated time for tumors to grow to 2000 mm³ after radiation therapy and subsequent EO9 or vehicle administration.

FIG. 7 shows trend lines plotted from FIG. 6.

FIG. 8 shows the average tumor volume after radiation therapy and prior EO9 or vehicle administration.

FIG. 9 shows the linear regression and estimated time for tumors to grow to 2000 mm³ after radiation therapy and prior EO9 or vehicle administration.

### DEFINITION OF TERMS

Prior to setting forth the invention, it may be helpful to provide an understanding of the definitions of certain terms that will be used hereinafter:

The term “patient” includes any living organism having at least one tumor. The living organism can be any mammal, fish, reptile or bird. Mammals include, but are not limited to, primates, including humans, dogs, cats, goats, sheep, rabbits, pigs, horses and cows.

The terms “treatment” or “contributing to the treatment of” include preventing, retarding the progression or growth of, shrinking, or eliminating a solid tumor. As such, these terms include both medical therapeutic and/or prophylactic administration, as appropriate.

The term “instructional information” includes information accompanying a pharmaceutical product that provides a description of how to administer the product, the purpose of the product and/or the safety and efficacy data required to allow a physician, pharmacist or patient to make an informed decision regarding use of the product. The instructional information generally is regarded as the “label” for a pharmaceutical product and can be included as a product insert. Instructional information can come in many different forms including, without limitation, a paper insert, a c.d. rom or a link to a website containing the instructional information.

The term “prodrug” includes compounds that transform rapidly in vivo to a compound useful in the invention, for example, by hydrolysis. A thorough discussion of prodrugs is provided in Higuchi et al., Prodrugs as Novel Delivery Systems, Vol. 14, of the A.C.S.D. Symposium Series, and in Roche (ed.), Bioreversible Carriers in Drug Design, American Pharmaceutical Association and Pergamon Press, 1987.

The term “sensitize” means to make more sensitive to an effect.

The phrase “radiation therapies” includes radiation treatments administered to a patient that are separated by a period of time. The period of time separating the radiation therapies can be determined by a treating physician or veterinarian and can include, without limitation, minutes, hours, days, weeks, months or years. A given radiation therapy can be the same as or different from the radiation therapy immediately preceding or following it.

### DETAILED DESCRIPTION

This invention relates to the discovery that indoloquinones can sensitize tumors to radiation therapy thereby contributing to the treatment of various cancers. More specifically, indoloquinones can sensitize hypoxic cells within tumors to radiation therapies by targeting these cells and damaging their DNA. Importantly, indoloquinones achieve these effects with minimal normal cell and tissue toxicity.

Apaziquone (“EO9”; 3-hydroxymethyl-5-aziridinyl-1-methyl-2-(1 H-indole-4,7-dione)-prop-β-en-α-ol; IUPAC: 3-hydroxymethyl-5-aziridinyl-1-methyl-2-(1 H-indole-4,7-dione)-propenol) is one indoloquinone that is a novel analogue of mitomycin C. The basic mechanism of activation of EO9 is believed to be similar to that of other indoloquinones, involving reduction by cellular enzymes that transfer one or two electrons, forming semiquinone and hydroquinone, respectively. Oxidation of the semiquinone under aerobic conditions results in a redox cycle that can cause cell death by forming reactive oxygen species (ROS), resulting in DNA strand breaks. The semiquinone/hydroquinone can, particularly under hypoxic conditions, alkylate and crosslink DNA and other macromolecules, causing cell death.

The reductases expressed in tumors may play an important role in the selectivity of EO9. NQO1 (NAD(P)H: quinone oxidoreductase), a two electron reductase enzyme, may selectively target oxygenated cells, while one electron reducing enzymes such as Cytochrome P450 reductase may be more effective in targeting hypoxic cells. Loadman et al., 137 Br. J. Pharmacol. 701-709, 2002. Various embodiments according to the present invention are described in the following examples.

### EXAMPLE 1

### Effect of Tumor Volume on Tumor Oxygen Tension

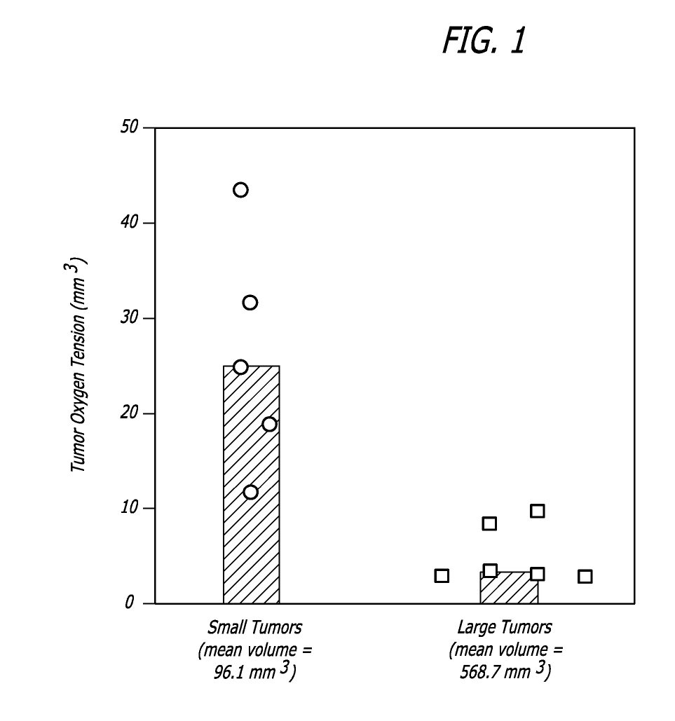

U-87 human glioblastoma cells (American Type Culture Collection) were maintained in alpha MEM medium (Sigma) with 10% fetal bovine serum (Atlanta Biologicals). U-87 human glioblastoma cells (5×10⁵ cells in 100 μl PBS) were injected subcutaneously into the right hind limb of athymic NCR NUM nude mice (Taconic Farms) and allowed to grow to a hypoxic volume of ˜550 mm³. Tumor oxygen tension was measured using the Oxford Oxylite fiberoptic probe (Oxford, England). The detection system is based on blue light excitation of ruthenium pigment at the end of a fiber optic probe, which is quenched by oxygen. Measurements were performed on anesthetized mice (75 mg/kg Ketamine and 0.3 mg/kg Acepromazine), while body temperature was maintained at 37° C. with a heating pad. A 25 gauge needle was used to puncture the tumor capsule to facilitate insertion of the fiberoptic probe. The probe was guided into the tumor at a 2-4 mm depth. FIG. 1 shows the median tumor pO₂ values for multiple small tumors (circles, N=5) and multiple large tumors (squares, N=6). Bars indicate group medians. The results indicate that small tumors have higher tumor oxygen tension than large tumors.

### EXAMPLE 2

### NQO1 and Cytochrome P450 Reductase Levels in Hypoxic Tumors

As described, U-87 human glioblastoma cells were injected subcutaneously into the right hind limb of athymic NCR NUM mice and allowed to grow to a diameter of ˜550 mm³ to induce hypoxia. Tumor samples were prepared in LDS Sample Buffer (Invitrogen, Carlsbad, Calif.) containing 40 mM dithiothreitol, 14 mg/L aprotinin, 0.7 mg/L pepstatin, and 5 mM 4-(2-aminoethyl)-benzenesulphonyl fluoride. Samples were resolved on NuPage 10% bis-Tris gels (Invitrogen, Carlsbad, Calif.). The proteins were transferred onto polyvinylidene difluoride membranes (Amersham Pharmacia Biotech, Piscataway, N.J.) using a semidry transfer apparatus (Pharmacia-LKB multiphor II). Immunoblotting was performed with monoclonal and polyclonal antibodies: anti-human NQO1, anti-human Cytochrome P450 reductase and anti-GAPDH. Immunodetection was performed by enhanced chemiluminescence using a Tropix Western-Star protein detection kit (Applied Biosystems; Foster City, Calif.).

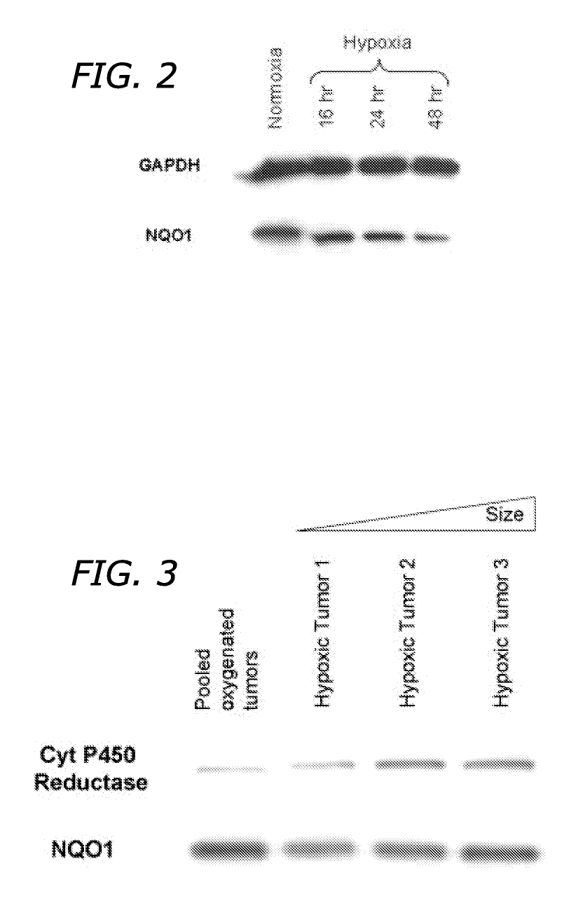

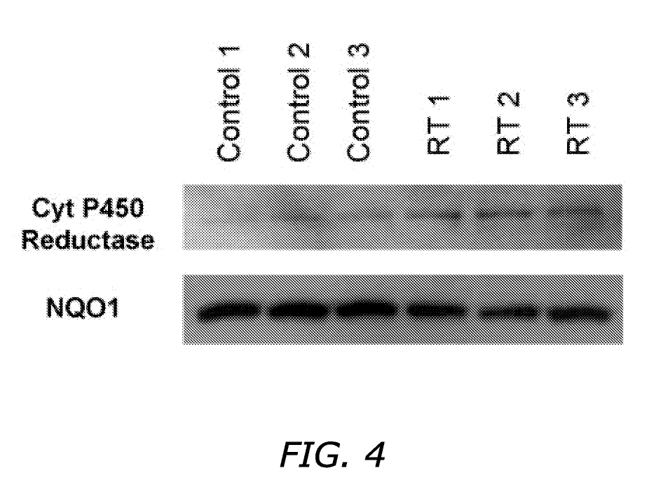

FIG. 2 shows western blot analysis of normoxic U87 cells and cells treated under hypoxia for increasing lengths of time. This FIG. 2 shows that hypoxia down regulates NQO1 in U87 tumor cells. FIG. 3 shows pooled tumor samples from three small tumors (lane 1; mean=143 mm³), and three hypoxic tumors of increasing size (lanes 2, 3 and 4; mean=693 mm³). This FIG. 3 shows that hypoxic tumors have a higher ratio of cytochrome P450 reductase to NQO1. FIG. 4 shows western blot analyses of three untreated tumors (Control 1, 2, 3) and three tumors (RT 1, 2, 3) that were irradiated with three daily fractions of 7.5 Gy. Samples were harvested 24 hours after the last irradiation. This FIG. 4 shows that fractionated radiation therapy increases the ratio of cytochrome P450 reductase to NQO1 in tumor xenografts. As stated earlier, and without being bound by theory, these differences in NQO1 and cytochrome P450 reductase levels may provide the basis for EO9's selectivity for hypoxic tumor cells.

### EXAMPLE 3

### Effect of EO9 & Radiation Therapy on Average Tumor Volume

### Example 3a

### Short Term Study

U-87 human glioblastoma cells (American Type Culture Collection) were maintained in alpha MEM medium (Sigma) with 10% fetal bovine serum (Atlanta Biologicals). A U-87 cell suspension was injected subcutaneously into the right hind limb (5×10⁵ cells in 100 μl PBS) of athymic NCR NUM mice (Taconic Farms) and allowed to grow to a hypoxic volume of ˜550 mm³ before treatment. Tumors were treated with EO9 and a fractionated radiation therapy schedule to test the hypothesis that EO9 would sensitize tumor cells to radiation therapy. EO9 or vehicle was administered 30 minutes after each radiation therapy fraction on day 1, 2, and 3. The study used 4 treatment groups: vehicle (DMSO), radiation therapy alone (3 days×7.5 Gy), EO9 (3 days×2 mg/kg), and EO9 +radiation therapy (3 days×7.5 Gy×2 mg/kg). EO9 was administered locally by intra-tumoral injection to achieve optimal delivery.

Irradiations were performed on anesthetized mice using an X-ray machine (Pantak) operating at 250 kV, 10 mA, with a 2-mm aluminum filtration. The effective photon energy was 90 keV. Mice were anesthetized with a combination of Ketamine and Acepromazine at a concentration of 75 mg/kg and 0.30 mg/kg respectively. Each mouse was confined in a lead casing with its tumor-bearing leg extended through an opening on the side to allow the tumor to be irradiated locally.

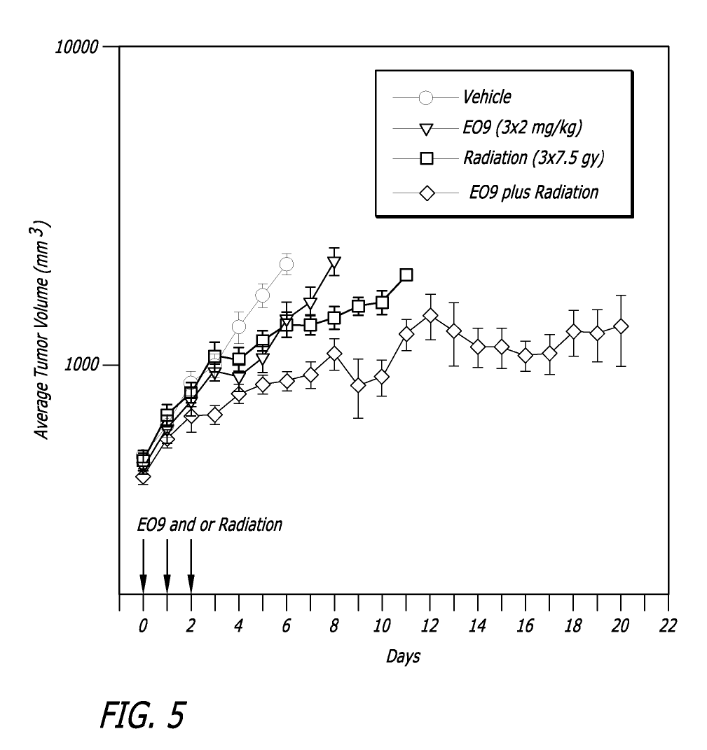

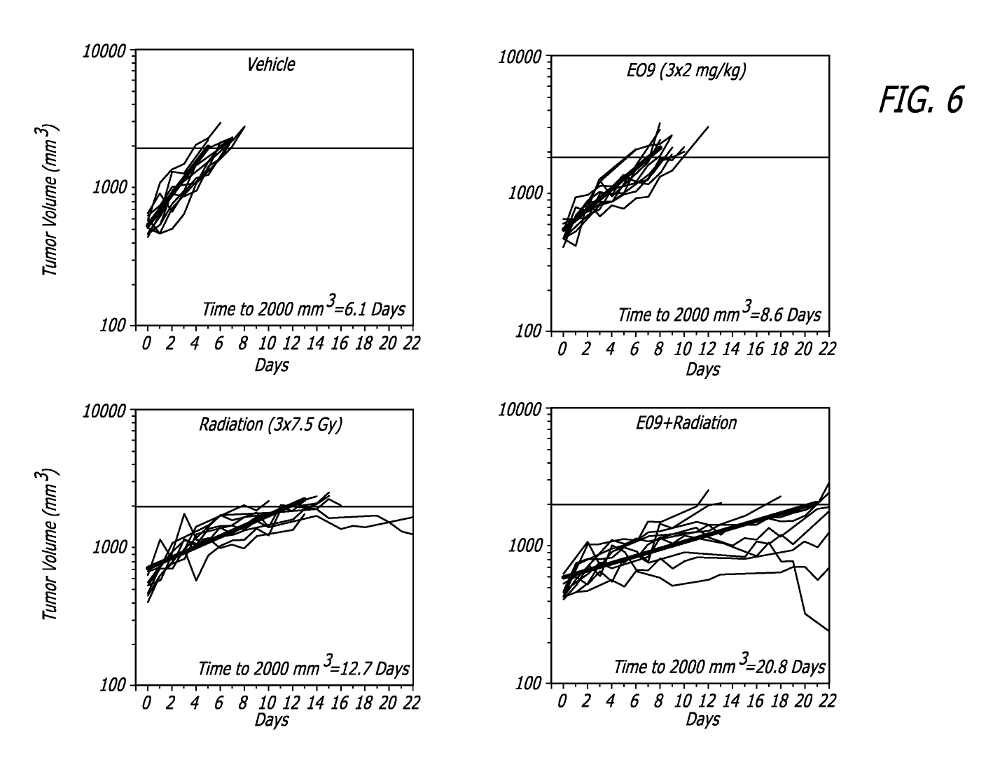

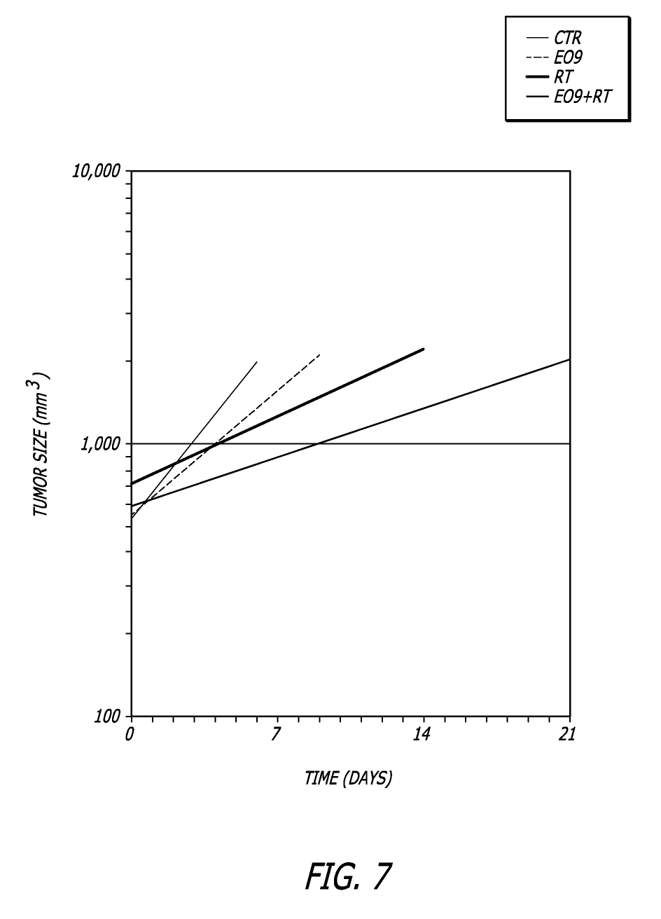

FIG. 5 shows the observed mean and standard error of the 4 treatment groups. Mixed-effects regression was used to model the base-10 logarithm of tumor volume as a function of time and treatment (tumor growth analyses). The log-transformed outcome was used because tumors of this size grow approximately exponentially, and therefore the logarithm of the tumor volume is approximately linear over time. This approach appropriately handles unbalanced data, such as a different number of measurements for different animals, and takes into account the correlation of each animal's measurements over time. These analyses were carried out with SAS 8.2 (SAS Institute Inc., Cary, N.C., 1999-2001). FIG. 6 shows linear regression and estimated time for tumors to grow to 2000 mm³ in animals receiving EO9 or vehicle 30 minutes after radiation therapy on day 1, 2, and 3. FIG. 7 shows the Trend lines plotted together from FIG. 6. As can be seen from these FIGS., animals receiving EO9 and radiation therapy showed the slowest rate of tumor growth.

As shown in the following Table, the average tumor growth in the vehicle treated group corresponded to a doubling time of 3.2 days. EO9 alone or radiation therapy alone when administered increased tumor doubling time to approximately 4.6 days (p<0.001 vs. control) or 8.4 days (p<0.001 vs. control), respectively. Combination of EO9 and radiation therapy increased the mean doubling time to 11.7 days, a stronger effect than that seen by a comparable regimen of E09 alone (p<0.001), or radiation therapy alone (p=0.027 comparing days 1-7). Therefore, the combination of EO9 and radiation therapy was additive. Additionally, no increase in weight loss or local normal toxicity was observed in any group after treatment with EO9.

| | | | Estimated Time |
|---|---|---|---|
| | | | (Days) for tumors |
| Treatment Group | % Δ (95% CI) | T₂ₓ | to reach 2000 mm³ |
| VEHICLE | 25 (21, 28) | 3.2 | 6.1 |
| EO9 | 16 (14, 19) | 4.6 | 8.6 |
| RT | 9 (6, 11) | 8.4 | 12.7 |
| EO9 + RT | 6 (4, 8) | 11.7 | 20.8 |

% Δ: average rate of increase of tumor volume (% daily increase).
95% CI: 95% confidence interval.
T₂ₓ: average doubling time of tumor volume (in days).

### Example 3b

### Long Term Study

A protocol was investigated where EO9 and radiation therapy were administered over a longer period of time. In this study, EO9 or radiation therapy was delivered alone or in combination on two non-consecutive days a week for 3 weeks. Six groups received vehicle alone (DMSO), vehicle 30 minutes before radiation therapy (2 days×7.5 Gy), vehicle 30 minutes after radiation therapy (2 days×7.5 Gy), E09 only (2 days×3 mg/kg), EO9 minutes before radiation therapy, and EO9 30 minutes after radiation therapy. EO9 was administered locally by intra-tumoral injection to achieve optimal delivery.

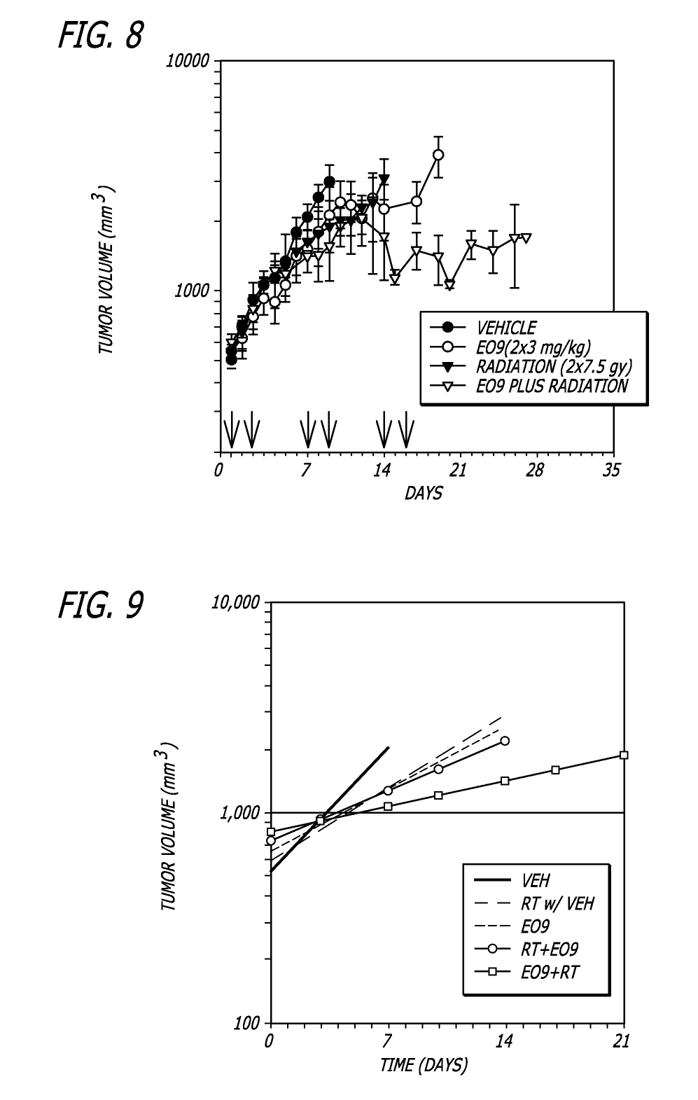

FIG. 8 shows the observed mean and SE for EO9 administered before or after radiation therapy for 2 days a week for 3 weeks. FIG. 8 combines the groups receiving EO9 before or after radiation therapy into a single group and shows that collectively this group showed the slowest rate of tumor volume growth. The following table which does not combine the two groups receiving EO9 before or after radiation therapy shows that the most effective dosing schedule was when EO9 was administered before radiation therapy. This dosing schedule resulted in the longest doubling time. The estimated times for tumors to reach 3000 mm³ include:

| Treatment Group | % Δ | (95% CI) | T_{2X} |
|---|---|---|---|
| VEH | 21 | (16, 27) | 3.6 |
| RT + VEH/VEH + RT | 10 | (8, 13) | 7.0 |
| E09 | 12 | (8, 16) | 6.1 |
| RT + E09 | 8 | (5, 11) | 9.1 |
| EO9 + RT | 4 | (1, 8) | 17.3 |

% delta: average rate of increase of tumor volume (% daily increase).
95% CI: 95% confidence interval.
T₂ₓ: average doubling time of tumor volume (in days).

When EO9 was delivered in a longer term regimen (twice a week for 3 weeks), EO9 was more effective when administered prior to radiation therapy. These studies confirm previous finding that EO9 has anti-tumor activity as a single agent and more importantly, demonstrate that EO9 is a significant radiation therapy sensitizer. FIG. 9 shows the linear regression and estimated time for tumors to grow to 2000 mm³ in animals receiving EO9 (3 mg/kg) or vehicle 30 minutes before or after radiation therapy (2 non-consecutive days/week×7.5 Gy) for three weeks.

### Summary of Results Described in Examples

Tumors averaging ˜550 mm³ were in the radiobiologically hypoxic range (FIG. 1). Hypoxia and fractionated radiation therapy increased the ratio of Cytochrome P450 reductase to NQO1, which could function to sensitize hypoxic tumors. (FIGS. 2-4). However, the presence of NQO1 may also function to eradicate oxygenated tumor cells in addition to radiation therapy. As shown in FIGS. 5, 6, 7, and Table 1, EO9 alone or radiation therapy alone increased tumor doubling time by 1.4 days (p<0.001 vs. control) or 5.2 days (p<0.001 vs. control), respectively. Combination of EO9 and radiation therapy increased the mean doubling time by 8.5 days to 11.7 days, a stronger effect than that seen by a comparable regimen of EO9 alone (p<0.001), or radiation therapy alone (p=0.027 comparing days 1-7). No significant increase in weight loss or normal toxicity was observed in any group after treatment with EO9. Additionally, a long-term treatment experiment was performed (FIGS. 8, 9 and Table 2). In the control group (vehicle), tumors had a doubling time of 3.6 days (Table 2). Radiation therapy alone substantially reduced this growth rate to 7.0 days (p=0.001 vs. control), as did EO9 alone to 6.1 days (p=0.006 vs. control). Radiation therapy in combination with EO9 led to further reductions in the tumor growth rate. Radiation therapy given before EO9 slowed the average daily tumor doubling time to 9.1 days (p=0.25 vs. radiation therapy alone, 0.11 vs. EO9 alone, and 0.001 vs. control). However, radiation therapy given after EO9 had the strongest effect, slowing the average daily tumor doubling time to 17.3 days (p=0.007 vs. radiation therapy alone, 0.005 vs. EO9 alone, and 0.001 vs. control). These results indicate for the first time that EO9 can benefit a fractionated regimen of radiation therapy and should be explored further as a radiation therapy sensitizer. EO9 alone or in combination with radiation therapy had statistically significant anti-tumor activity.

Pharmaceutical compositions containing the active ingredients according to the present invention are suitable for administration to humans or other mammals. Typically, the pharmaceutical compositions are sterile, and contain no toxic, carcinogenic, or mutagenic compounds that would cause an adverse reaction when administered. Administration of the pharmaceutical composition can be performed before, during, or after the onset of solid tumor growth.

A method of the present invention can be accomplished using active ingredients as described above, or as a physiologically acceptable salt, derivative, prodrug, or solvate thereof. The active ingredients can be administered as the neat compound, or as a pharmaceutical composition containing either or both entities.

The pharmaceutical compositions include those wherein the active ingredients are administered in an effective amount to achieve their intended purpose. More specifically, a “therapeutically effective amount” means an amount effective to prevent development of, to eliminate, to retard the progression of, or to reduce the size of a solid tumor. Determination of a therapeutically effective amount is well within the capability of those skilled in the art, especially in light of the detailed disclosure provided herein.

A “therapeutically effective dose” refers to that amount of the active ingredients that results in achieving the desired effect. Toxicity and therapeutic efficacy of such active ingredients can be determined by standard pharmaceutical procedures in cell cultures or experimental animals, e.g., determining the LD50 (the dose lethal to 50% of the population) and the ED50 (the dose therapeutically effective in 50% of the population). The dose ratio between toxic and therapeutic effects is the therapeutic index, which is expressed as the ratio between LD50 and ED50. A high therapeutic index is preferred. The data obtained can be used in formulating a range of dosage for use in humans. The dosage of the active ingredients preferably lies within a range of circulating concentrations that include the ED50 with little or no toxicity. The dosage can vary within this range depending upon the dosage form employed, and the route of administration utilized.

The exact formulation and dosage is determined by an individual physician in view of the patient's condition. Dosage amount and interval can be adjusted individually to provide levels of the active ingredients that are sufficient to maintain therapeutic or prophylactic effects. As stated, the methods, compositions and dosing regimens according to the present invention can be applied or administered before all radiation therapies of a patient; before a subset of a radiation therapies of a patient; after all radiation therapies of a patient; after a subset of a radiation therapies of a patient; before and after all a radiation therapies of a patient; before all radiation therapies of a patient and after a subset of a radiation therapies of a patient; before a subset of a radiation therapies of a patient and after all radiation therapies of a patient; and before a subset of a radiation therapies of a patient and after a subset of a radiation therapies of a patient.

The amount of pharmaceutical composition administered can be dependent on the subject being treated, on the subject's weight, the severity of the affliction, the manner of administration, and the judgment of the prescribing physician.

The active ingredients can be administered alone, or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of the active ingredients into preparations which can be used pharmaceutically.

When a therapeutically effective amount of the active ingredients is administered, the composition can be in the form of a pyrogen-free, parenterally acceptable aqueous solution. The preparation of such parenterally acceptable solutions, having due regard to pH, isotonicity, stability, and the like, is within the skill in the art.

For veterinary use, the active ingredients are administered as a suitably acceptable formulation in accordance with normal veterinary practice. The veterinarian can readily determine the dosing regimen that is most appropriate for a particular animal.

Various adaptations and modifications of the embodiments can be made and used without departing from the scope and spirit of the present invention which can be practiced other than as specifically described herein. The above description is intended to be illustrative, and not restrictive. The scope of the present invention is to be determined only by the claims.

The terms and expressions which have been employed herein are used as terms of description and not of limitation, and there is no intention in the use of such terms and expressions of excluding equivalents of the features shown and described, or portions thereof, it being recognized that various modifications are possible within the scope of the present invention claimed. Moreover, any one or more features of any embodiment of the present invention can be combined with any one or more other features of any other embodiment of the present invention, without departing from the scope of the present invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the present invention otherwise claimed. No language in the specification should be construed as indicating any non-claimed element essential to the practice of the present invention.

Groupings of alternative elements or embodiments of the present invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Certain embodiments according to the present invention are described herein, including the best mode known to the inventors for carrying out the invention. Of course, variations on these embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the present invention disclosed herein are illustrative of the principles of the present invention. Other modifications that may be employed are within the scope of the present invention. Thus, by way of example, but not of limitation, alternative configurations of the present invention may be utilized in accordance with the teachings herein. Accordingly, the present invention is not limited to that precisely as shown and described.

What is claimed is:

1. A method comprising sensitizing one or more tumors to radiation therapy by administering one or more indoloquinones.

2. A method according to claim 1, wherein said one or more tumors comprise hypoxic cells.

3. A method according to claim 1, further comprising administering said one or more indoloquinones to a patient in need thereof wherein said administering comprises systemic and/or local administration and said patient will receive at least two radiation therapies.

4. A method according to claim 3, wherein said administering of said one or more indoloquinones occurs through a route selected from one or more of the group consisting of oral administration, intra-tumoral administration, intravenous administration, intravesical administration and intraarterial administration.

5. A method according to claim 1 wherein said one or more indoloquinones comprises apaziquone (EO9).

6. A method according to claim 3 wherein said administering of said one or more indoloquinones occurs in a manner selected from the group consisting of before all radiation therapies of said patient; before a subset of said radiation therapies of said patient; after all radiation therapies of said patient; after a subset of said radiation therapies of said patient; before and after all said radiation therapies of said patient; before all radiation therapies of said patient and after a subset of said radiation therapies of said patient; before a subset of said radiation therapies of said patient and after all radiation therapies of said patient; and before a subset of said radiation therapies of said patient and after a subset of said radiation therapies of said patient.

7. A composition comprising one or more indoloquinones wherein said one or more indoloquinones are directed to be administered in conjunction with a radiation therapy for the treatment of a tumor.

8. A composition according to claim 7, wherein said tumor comprises hypoxic cells.

9. A composition according to claim 7, wherein said one or more indoloquinones are directed to be administered systemically and/or locally.

10. A composition according to claim 7, wherein said one or more indoloquinones are directed to be administered through a route selected from one or more of the group consisting of oral administration, intra-tumoral administration, intravenous administration, intravesical administration and intraarterial administration.

11. A composition according to claim 7 wherein said one or more indoloquinones comprise apaziquone (EO9).

12. A composition according to claim 7, wherein said one or more indoloquinones are directed to be administered to a patient who will receive at least two radiation therapies.

13. A composition according to claim 13, wherein said one or more indoloquinones are directed to be administered in a manner selected from the group consisting of before all radiation therapies of said patient; before a subset of said radiation therapies of said patient; after all radiation therapies of said patient; after a subset of said radiation therapies of said patient; before and after all said radiation therapies of said patient; before all radiation therapies of said patient and after a subset of said radiation therapies of said patient; before a subset of said radiation therapies of said patient and after all radiation therapies of said patient; and before a subset of said radiation therapies of said patient and after a subset of said radiation therapies of said patient.

14. A dosing regimen comprising one or more indoloquinones and instructional information that directs the administration of said one or more indoloquinones in conjunction with a radiation therapy for the treatment of a tumor.

15. A dosing regimen according to claim 14, wherein said tumor comprises hypoxic cells.

16. A dosing regimen according to claim 14, wherein said instructional information directs the systemic and/or local administration of said one or more indoloquinones.

17. A dosing regimen according to claim 14, wherein said instructional information directs the.administration of said one or more indoloquinones through a route selected from one or more of the group consisting of oral administration, intra-tumoral administration, intravenous administration, intravesical administration and intraarterial administration.

18. A dosing regimen according to claim 14 wherein said one or more indoloquinones comprise apaziquone (EO9).

19. A dosing regimen according to claim 14 wherein instructional information directs said one or more indoloquinones to be administered to a patient who will receive at least two radiation therapies.

20. A dosing regimen according to claim 19, wherein said one or more indoloquinones are directed to be administered in a manner selected from the group consisting of before all radiation therapies of said patient; before a subset of said radiation therapies of said patient; after all radiation therapies of said patient; after a subset of said radiation therapies of said patient; before and after all said radiation therapies of said patient; before all radiation therapies of said patient and after a subset of said radiation therapies of said patient; before a subset of said radiation therapies of said patient and after all radiation therapies of said patient; and before a subset of said radiation therapies of said patient and after a subset of said radiation therapies of said patient.
